(19) United States
(12) Patent Application Publication
(10) Pub. No.: US 2023/0162305 A1
(43) Pub. Date: May 25, 2023

(54) INFORMATION SYSTEM

(71) Applicants: HITACHI, LTD., Chiyoda-ku, Tokyo (JP); KYOTO UNIVERSITY, Kyoto-shi, Kyoto (JP)

(72) Inventors: Takeshi KATO, Kyoto (JP); Yasuo DEGUCHI, Kyoto (JP); Yasuyuki KUDO, Tokyo (JP); Junichi MIYAKOSHI, Tokyo (JP); Seiji MIURA, Tokyo (JP); Misa MIYAKOSHI, Tokyo (JP); Yasuhiro ASA, Tokyo (JP); Hiroyuki MIZUNO, Tokyo (JP)

(21) Appl. No.: 17/920,119

(22) PCT Filed: Apr. 24, 2020
(86) PCT No.: PCT/JP2020/017663

§ 371 Date: Oct. 20, 2022

(51) Int. Cl. G06Q 50/22; A61B 5/16; G06Q 50/26

(52) U.S. Cl. CPC G06Q 50/22; A61B 5/165; A61B 5/167; G06Q 50/26

(57) ABSTRACT

A social system operates based on continuous consensus building regarding the selection of diverse values.

An information system includes a collection unit that collects first data regarding individual behavior, second data regarding interpersonal interaction, and third data regarding the current institution in a social group; a first system that diagnoses at least one of the first data and the second data under the current institution and intervenes in at least one of the individual behavior and the interpersonal interaction based on the diagnosis result; and a second system that generates a plurality of options for a future institution in the social group based on the updated at least one of the first data and second data, and the third data, supports consensus building of the social group on the plurality of options, and updates the institution of the social group based on options agreed upon by the social group.

### TECHNICAL FIELD

The present invention relates to an information system.

### BACKGROUND ART

The economy is the activity in which human society acquires resources from the natural environment and produces, exchanges, distributes, and consumes the goods and services necessary' for human life. In the related art, various studies such as welfare economics, normative economics, comparative institutional analysis, and the like have been conducted toward the desirable economic system for human society.

The information systems that support economic actives can be roughly divided into centralized systems and distributed systems. A centralized system is, for example, a cloud platform. A distributed system is, for example, a distributed network. Many system operation techniques are known for both centralized and distributed. systems.

According to sociologist Talcott Parsons, social systems have four functional prerequisites (adaptation, goal attainment, integration, and pattern maintenance), and corresponding intervention methods for social integration include four strategies (convenience, power, persuasion, and enlightenment),

Technologies that support consensus building in a social group include communication support types such as video conferences and chats, process support types such as topic extraction and argument analysis, and negotiation support types such as alternative proposals and trade-off analysis. Further, for example, JP-A-2007-323526 (PTL 1) describes an environmental control system that performs control based on a subject's request by a consensus building control device in controlling environmental equipment.

### CITATION LIST

### Patent Literature

PTL 1: JP-A-2007-323526

### SUMMARY OF INVENTION

### Technical Problem

The SDGs (Sustainable Development Goals) proposed by the United Nations are set for the goals related to eradicating poverty and hunger, correcting income disparities and inequalities, securing energy and preserving the environment, inclusive employment, and institutions, and aim to create a sustainable, diverse, and inclusive society that leave no one behind.

In addition, the Cabinet. Office of Japan has proposed “a human-centered society (Society 5.0) that achieves both economic development and the resolution of social issues through a system that highly integrates cyber space (virtual space) and physical space (real space)” as a concept for the future society following the hunting society (Society 1.0), the agricultural society (Society 2.0), the industrial society (Society 3.0), and the information society (Society 4.0).

In order to resolve social issues such as disparities and inequalities listed in the SDGs, global warming and environmental pollution, and to realize a sustainable, fair, and inclusive society, we cannot avoid considering social norms in a broad sense, including fairness, equity, goodness, justice, duty, morals, ethics, and the like. Therefore, combining the perspective of the fusion of society and IT (Information. Technology) and the human-centered perspective, in Society 5.0, the IT system diagnoses and predicts the prognosis of the social system, and based on the results, it will make real-time and dynamdc normative interventions into the social system. In other words, it is necessary to incorporate norms and ethics a priori into information systems.

By the way, a social system. requires both operation (administration and judiciary) and consensus building (legislation and politics). According to the philosopher Yasuo Deguohi, society is a manifold of values, and in order to maintain social order while maintaining and operating the diversity of indivduals, it is necessary to continuously build a consensus in groups.

In addition, with the spread of CSV (Creating Shared Value) and ESG (Environment, Social, and Governance) in recent economic activities, in consensus building, not only economic value but also environmental value related to nature and resources, and social value related to life and culture should also be considered.

However, the related art information system operation technology is limited to optimizing and maximizing only economic utility and economic value, and consideration. has not been given to non-economic social and environmental values such as social norms, ethics, environment, and culture.

In addition, in the related intervention technology, the subject's behavior plan is set in advance from the outside rather than being determined by the subject himself/herself, the related consensus building support technology is separated from the operation of the economic system, and no consideration is given to follow-up to changes in economic situations and environmental conditions over time and continuous consensus building.

More specifically, for example, in the consensus building control device described in PTL 1, although the demand of the subject is reflected in the content of control, the consensus building rule itself representing the content of the control is set in advance from the outside rather than being based on the agreement with the subject.

The present invention has been made in view of this circumstance, and in a co-operating system between a social system and an information system, an object thereof is to provide a technology to operate in incorporation. with not only economic utility but also social norms and ethics, and to perform continuous consensus building on the selection of various social, environmental, and economic values according to changes in. economic situations and environmental conditions.

### Solution to Problem

The present application includes a plurality of means for solving at least part of the above problems, and the following are examples of such means.

In order to solve the above problems, an information system according to an aspect of the present invention is characterized to include a first system including a collection unit that collects first data regarding individual behavior, second data regarding interpersonal interaction, and third data regarding the current institution in a social group, an individual psychology diagnosis unit that diagnoses at least one of the first data and the second data under the current institution, and an intervention unit that updates at least one of the first data and the second. data by intervening in at least one of the individual behavior and the interpersonal interaction based on a diagnosis result on the at least one of the first data and the second data; and a second system including an option generation unit that generates a plurality of options for a future institution in the social group based on the updated at least one of the first data and the second data, and the third data, a consensus building unit that supports consensus building in the social group for the plurality of generated options, and an update unit that updates the institution in the social group based on the options agreed upon by the social. group.

### Advantageous Effects of Invention

According to one aspect of the present invention, in a co-operating system between a social system and an information system, it is possible to provide a technology to operate in incorporation with not only economic utility but also social norms and ethics, and to perform continuous consensus building on the selection of various social, environmental, and economic values according to changes in economic situations and environmental conditions.

Problems, configurations, and effects other than those described above will be clarified by the following description of the embodiments.

### BRIEF DESCRIPTION OF DRAWINGS

FIG. 1 is a schematic diagram showing a configuration. example of a co-operating system according to an embodiment of the present invention.

FIG. 2 is a flowchart for illustrating an example of the processing by the co-operating system.

FIG. 3 is a diagram showing a configuration example of a fast system,

FIG. 4 is a diagram illustrating the processing of a collection unit in the fast system.

FIG. 5 is a diagram for illustrating the processing of an individual psychology diagnosis unit in the fast system.

FIG. 6 is a diagram illustrating the processing of an intervention unit in the fast system.

FIG. 7 is a diagram showing a configuration example of a slow system,

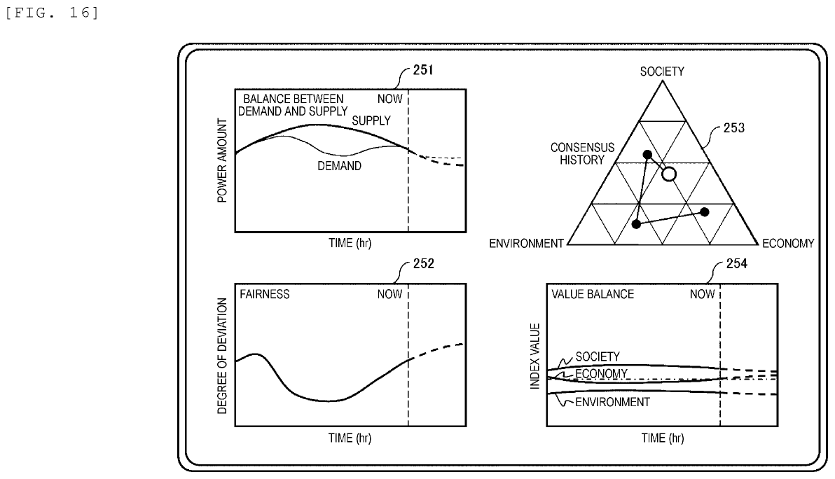

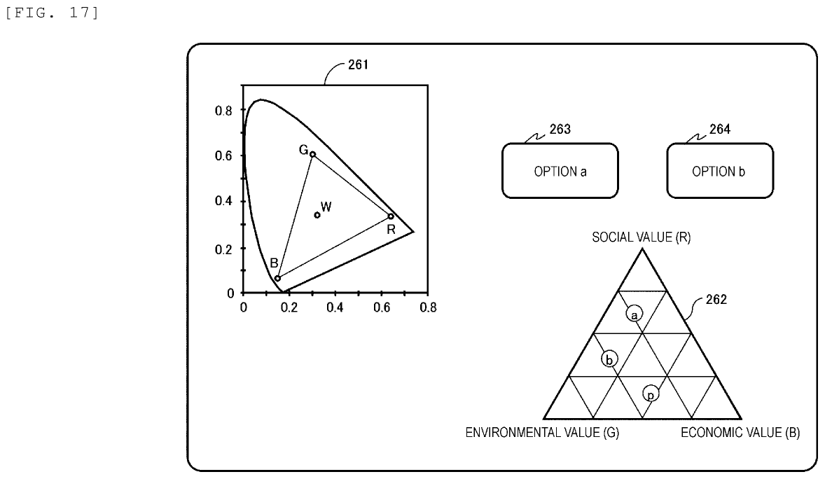

FIG. 6 is a diagram illustrating the processing of a collection unit and a group state diagnosis unit in the slow system.

FIG. 9 is a diagram illustrating the processing of a prognosis prediction unit in the slow system.

FIG. 10 is a diagram illustrating the processing of an option generation unit. in the slow system.

FIG. 11 is a diagram illustrating the processing of a consensus building unit in the slow system.

FIG. 12 is a diagram showing a display example on a user I/F of an IoH device.

FIG. 13 is a diagram showing a display example on the user I/F of the IoH device.

FIG. 14 is a diagram showing a display example on the user I/F of the IoH device.

FIGS. 15A-15C are examples of a user I/F other than a display in the IoH device, FIG. 15A is an example of a level meter, FIG. 15B is an example of a smart speaker, and FIG. 15C is a diagram showing an example of a smart light.

FIG. 16 is a diagram showing a display example on the user I/F of a server.

FIG. 17 is a diagram showing another display example on the user I/F of the IoH device.

FIG. 18 is a diagram showing yet another display example on the user I/F of the IoH device.

FIG. 19 is a conceptual diagram showing an application example of the co-operating system.

### DESCRIPTION OF EMBODIMENTS

emboddment of the present invention will be described below with reference to the drawings. In principle, the same members are denoted by the same reference numerals in all the drawings for illustrating the present embodiment, and the repeated descriptjon thereof will be omitted In addition, in the following embodments, it is needless to say that the constituent elements (including element steps, etc.) are not necessarily essential, unless otherwise explicitly stated or clearly considered. essential in principle. In addition, when saying “composed of A”, “consisting of A”, “having A”, or “including A”, it is needless to say that other elements are not excluded unless clearly stated that there is only that element. Similarly, in the following emboddments, when referring to the shape, posonal relationship, and the like of constituent elements, and the like, unless otherwise explicitly stated or in principle clearly considered to be otherwise, the actual shape shall include those that are comparable or similar to and the like.

<Configuration Example of Co-operating System 1 According to One Embodiment of Present Invention>

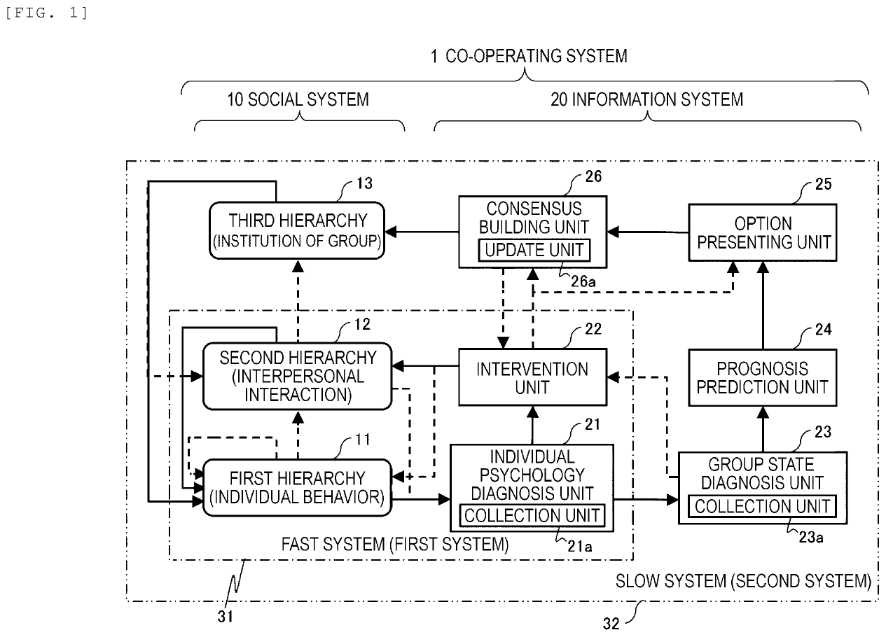

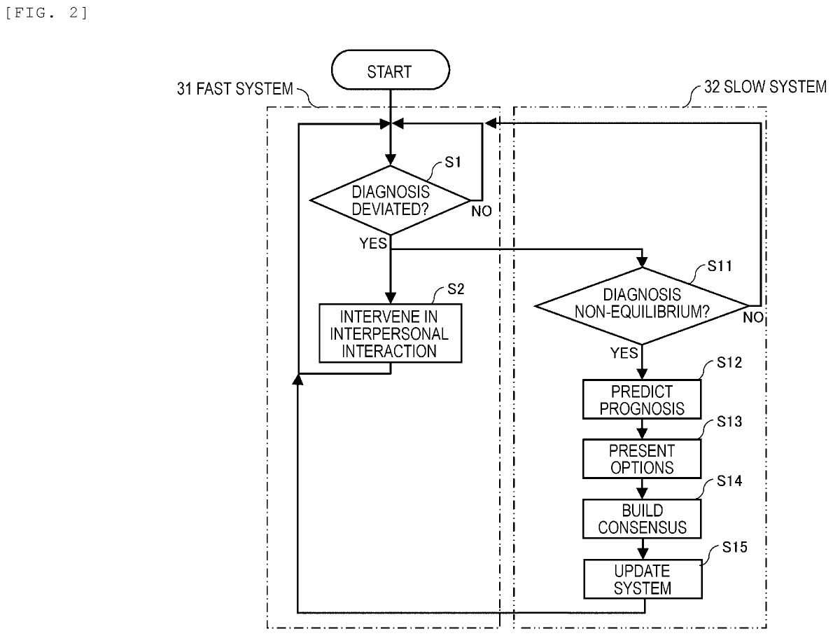

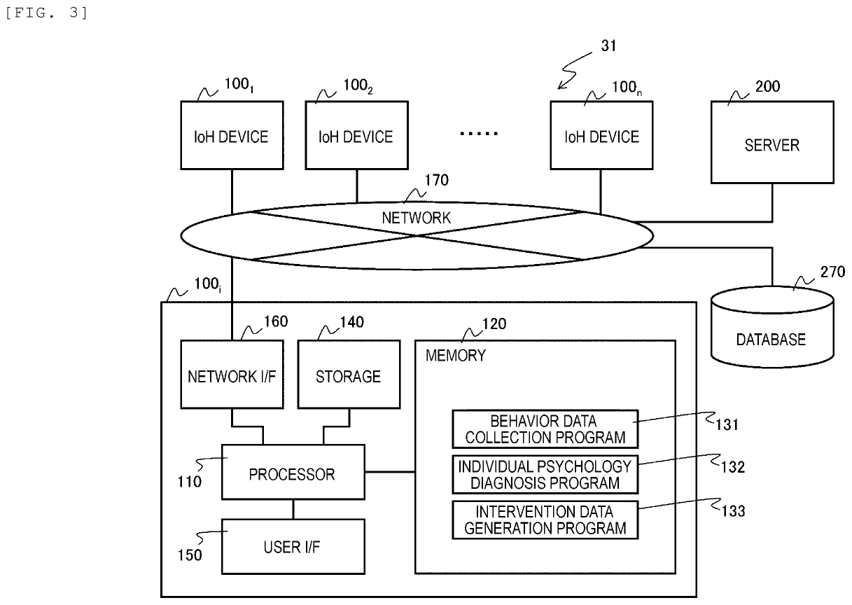

FIG. 1 is a schematic diagram showing an example of a co-operating system 1 according to one embodiment of the present invention.

The co-operating system 1 consists of a social system 10 and an information system 20. Also, from another point of view, the co-operating system 1 consists of a fast system 31 and a slow system 32. In the drawing, the fast system 31 is indicated by a one-dot chain line, and the slow system 32 is indicated by a two-dot chain. line. The fast system 31 corresponds to a first system of the present invention. The slow system 32 corresponds to a second system of the present invention.

The social system 10 has three hierarchies: a first hierarchy 11 related to individual behavior, a second hierarchy 12 related to interpersonal interaction, and a third hierarchy 13 related to an institution of a social group which is a collection of individuals. In the social system 10, a recursive loop is formed, in which mainly the first hierarchy 11 affects the second hierarchy 12, the second hierarchy 12 affects the third. hierarchy 13, and the third hierarchy 13 affects the first hierarchy 11.

Hereinafter, the first hierarchy 11 may be referred to as an individual behavior 11, the second hierarchy 12 as an interpersonal interaction 12, and the third hierarchy 13 as an institution 13.

The information system 20 consists of functional blocks of an individual psychology diagnosis unit 21, an intervention unit 22, a group state diagnosis unit 23, a prognosis prediction unit 24, an option generation unit 25, and a consensus building unit 26.

Of the functional blocks that constitute the information system 20, the individual psychology diagnosis unit 21 and the intervention unit 22 belong to the fast system 31.

The individual psychology diagnosis unit 21 includes a collection unit 21a. The collection unit 21a collects first data regarding individual behavior, second data regarding interpersonal interaction, and third data regarding the operation of a social group. The individual psychology diagnosis unit 21 diagnoses the individual behavior 11 or the interpersonal interaction 12 under the current institution 13 based on the first to third data collected by the collection unit 21a

The intervention unit 22 intervenes in the individual behavior 11 or the interpersonal interaction 12 based on the diagnosis result by the individual psychology diagnosis unit 21. Here, “intervention” refers to an operation or the like calling attention to correct the individual behavior 11 or the like, for example.

The fast system 31 forms a first circulation loop mainly in the order of the individual behavior 11, the individual psychology diagnosis unit 21, the intervention unit 22, and the interpersonal interaction 12, and operates the social system 10 in a responsive manner.

Of the functional blocks that constitute the information system 20, the group state diagnosis unit 23, the prognosis prediction unit 24, the option Generation unit 25, and the consensus building unit. 26 belong to the slow system 32.

The group state diagnosis unit 23 includes a collection unit 23a. The collection unit 23a collects operation data of the fast system 31. Here, the operation data of the fast system 31 refers to the updated first data and second data, and the third data The group state diagnosis unit 23 diaGnoses the current state of a group, which is a collection of individuals, and predicts the future state of the group, based on the operation data of the fast system 31.

The prognosis prediction unit 24 predicts prognosis based on the diagnosis results of the group state diagnosis unit 23. The option generation unit 25 generates options as the future institution based on the prognosis prediction. The consensus building unit 26 presents the options generated by the option. generation unit 25 to each individual, thereby supporting consensus building of a social group, which is a collection of individuals. The consensus building unit 26 includes an update unit 26a. The update unit 26a updates the current institution 13 based on options agreed upon by the social group. Here, the update refers to, for example, an operation of partially changing the institution 16 or completely revising the institution 16.

The slow system 32 forms a deliberative second circulation loop mainly in the order of the individual behavior 11, the individual psychology diagnosis unit 21, the group state diagnosis unit 23, the prognosis prediction unit 24, the option generation unit 25, the consensus building unit 26, and the institution 13, and continuously updates the institution 13 in the social system 10.

In FIG. 1, the first circulation loop by the fast system 31 and the second circulation loop by the slow system 32 are indicated by solid line arrows for easy viewing, and the other loops are indicated by dotted line arrows.

The fast system 31 allows intervention in the individual behavior 11 or the interpersonal interaction 12 in the light of currentinstitution 13 that reflects prior consensuses, and thus, the social system 10 can be operated in consideration of social norms as well as economic utility.

According to the slow system 32, since options for the future institution are presented based on the state of the social group under the current institution 13, and the institution 13 is updated after the consensus building of the social group, it is possible to update the institution 13 in consideration of not only the economy but also various social values such as the environment and culture while improving followability to relatively large changes in economic situations and environmental conditions.

Therefore, through cooperation between the fast system 31 and the slow system 32, the social system 10 can be normatively operated, and the institution 13 can be continuously maintained and updated.

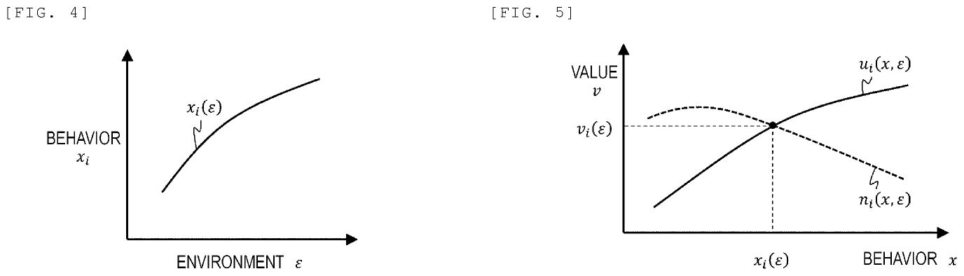

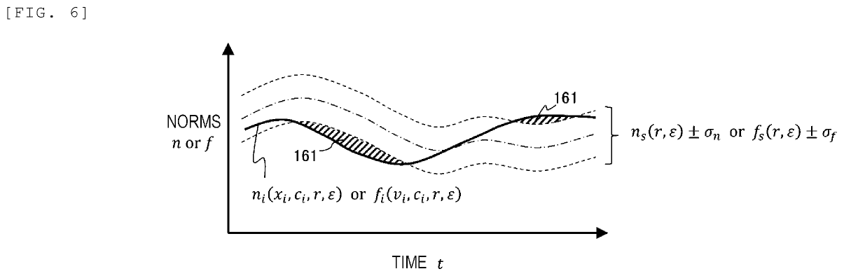

Next, FIG. 2 is a flowchart for illustrating an example of the processing by the co-operating system 1.

The processing is started in response to a predetermined start operation from the user, and then continuously repeated until a predetermined end operation is performed.

First, the collection unit 21a collects the first data regarding the individual behavior 11, and the individual psychology diagnosis unit 21 determines whether the individual behavior 11 deviates from a predetermined standard based on the first data (step S1). Here, if it is determined that the individual behavior 11 does not deviate from the predetermined standard (NO in step S1), the diagnosis of the individual behavior 11 is repeated.

On the contrary, if it is determined that the individual behavior 11 deviates from the predetermined standard (YES in step S1), the fast system 31 and the slow system 32 advance the respective processes.

In the fast system 31, the intervention unit 22 next intervenes in the interpersonal interaction 12 based on the diagnosis result for the individual behavior 11 (step S2). After this, the process returns to step S1, and the diagnosis for the individual behavior 11 affected by the intervening interpersonal interaction 12 is recursively performed.

Meanwhile, in the slow system 32, the collection unit 23a collects the operation data of the fast system 31, the group state diagnosis unit 23 diagnoses the state of the group based on the operation data of the fast system 31, and determines whether or not the current. state of the group is in non-equilibrium (step S11). Here, if it is determined that the current state of the group is not in non-equilibrium, that is, the current state of the group is in equilibrium (NO in step S11), the process returns to step S1, and step S1 and subsequent steps are repeated.

On the contrary, if it is determined that the state of the group is in non-equilibrium (YES in step S11), the prognosis prediction unit 24 predicts prognosis based on the diagnosis result of the group state diagnosis unit 23 (step S12). Next, the option generation unit 25 generates options for updating the current institution 13 to a future institution based on the prognosis prediction (step S13). Next, the consensus building unit 26 presents the options generated by the option generation unit 25, and supports group consensus building on the options (step S14). Next, the update unit 26a updates the current institution 13 based on the agreed options based on the consensus of the group (step S15). After this, diagnosis for the individual behavior 11 under the updated institution 13 is performed recursively.

<Configuration Example of Fast System 31>

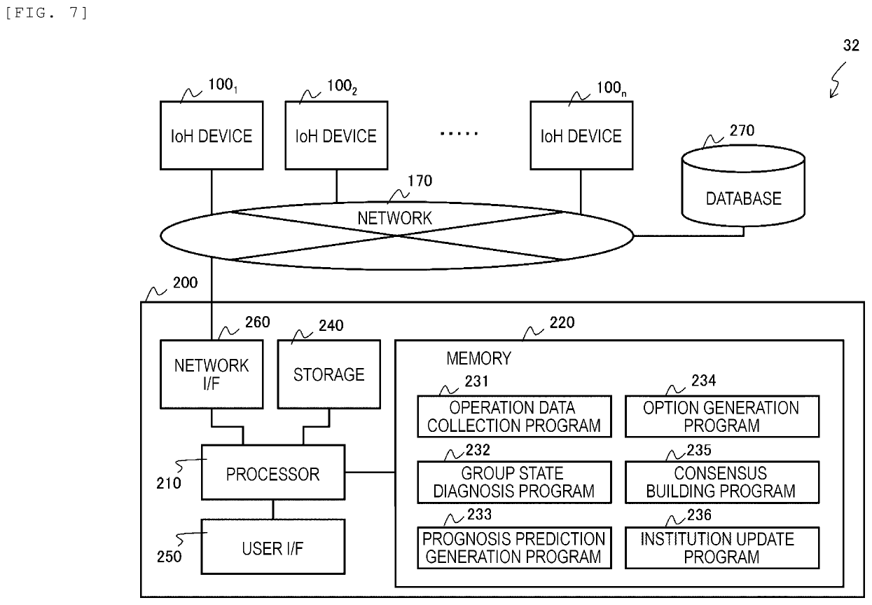

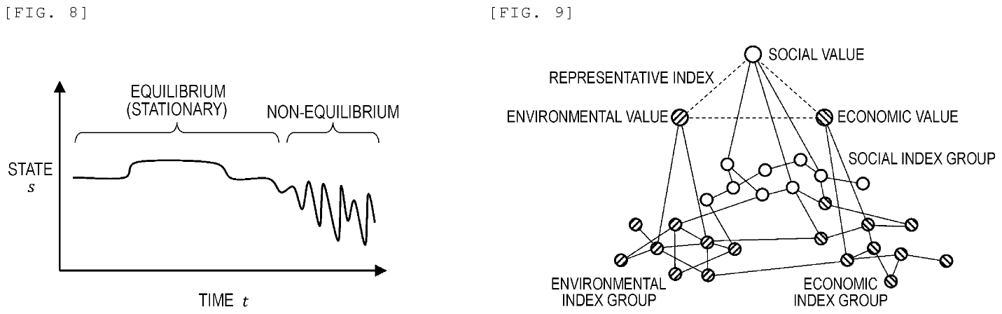

Next, FIG. 3 shows a configuration example of the fast system 31. The fast system 31 is composed of a plurality of IoH (Internet of Human) devices 100₁ to 100ₙ connected via a network 170. Hereinafter, the IoH devices 100₁ to 100ₙ are simply referred to as the IoH device 100 if individual distinguishment is not necessary. Further, the IoH device 100 used by an individual i will be referred to as the IoH device 100ᵢ as required.

The IoH device 100 is a network communication function added to various types of wearable products such as watches, eyeglasses, clothes, and the like, in addition to electronic devices represented by smartphones and tablet computers.

The IoH device 100 collects behavior data xᵢ of the individual i. The IoH device 100 also collects the characteristics cᵢ of the individual i, the data of the environment ε, and the like.

The IoH device 100 connects to a server 200 and a database 270 via the network 170.

The IoH device 100 includes a processor 110 such as a CPU (Central Processing Unit) and the like, a memory 120 such as a DRAM (Dynamic Random Access Memory) and the like, a storage 140 such as an HDD (Hard Disc Drive), an SSD (Solid State Drive), and the like, a user I/F (interface) 150 such as a touch panel, display, and the like, and a network I/F 160 such as a communication module and the like.

In the IoH device 100, a behavior data acquisition program 131, an individual psychology diagnosis program 132, and an intervention data generation program 133, which are stored in the storage 140, are read out to the memory 120 and executed by the processor 110, thereby implementing the collection unit 21a, the individual psychology diagnosis unit 21, and the intervention unit 22 belonging to the fast system 31 among the functional blocks constituting an information system 20.

Here, it is assumed that the fast system 31 consisting of a plurality of IoH devices 100 is applied to a distributed cooperative system that operates shared resources.

An individual i obtains value by determining his/her own resource usage in light of the current institution 13 while making mutual comparisons through communication with others j (≠i) in the interpersonal interaction 12. In the IoH device 100ᵢ, the collection unit 21a collects the behavior data of the individual i, the individual psychology diagnosis unit 21 diagnoses the psychology of the individual i based on the behavior data of the individual i, and the intervention unit 22 intervenes in the individual i by displaying a screen of a display as the user I/F 150, thereby cooperation with the group of individuals regarding the use of shared resources is encouraged.

Next, the processing of the collection unit 21a, the individual psychology diagnosis unit 21, and the intervention unit 22 belonging to the fast system 31 will be described in detail.

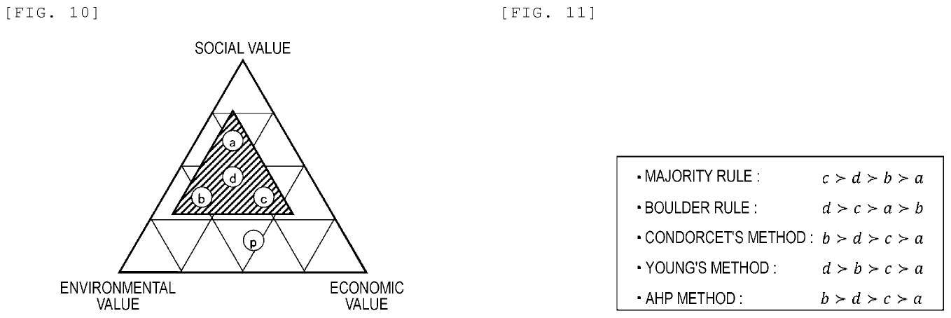

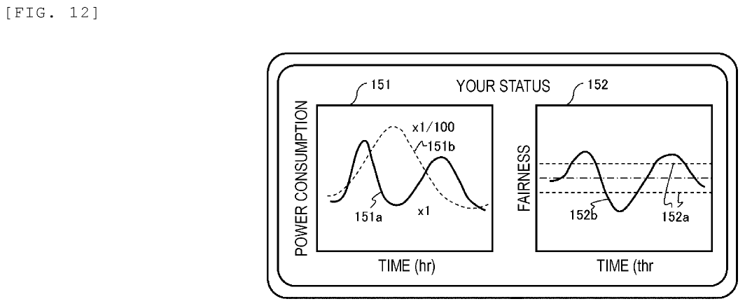

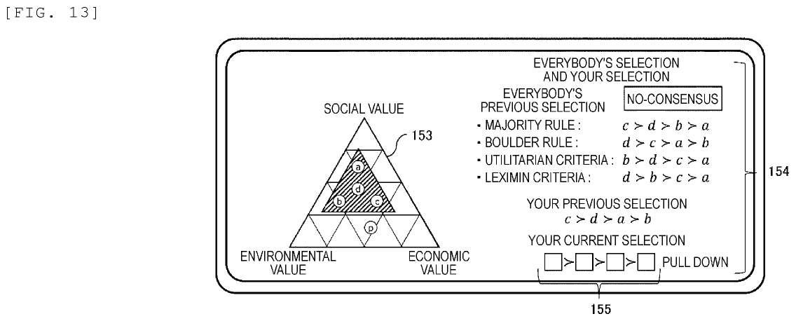

FIG. 4 is a diagram illustrating the processing of the collection unit 21a. The collection unit 21a collects from the individual behavior 11 data representing the behavior xᵢ (ε) of the individual i that changes according to the change in the environment 2 as shown in the drawing. Social norms are enabled by mutual recognition between individuals, and the behavior xᵢ (ε) of the individual i is affected by social norms. Therefore, the behavior xᵢ (ε) of the individual i can be regarded as indirectly reflecting the interpersonal interaction 12.

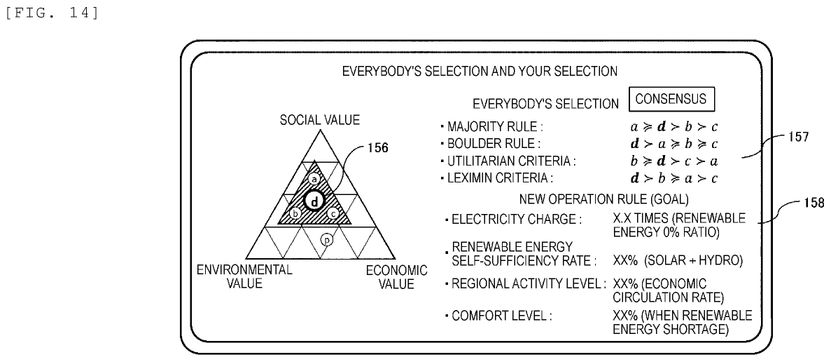

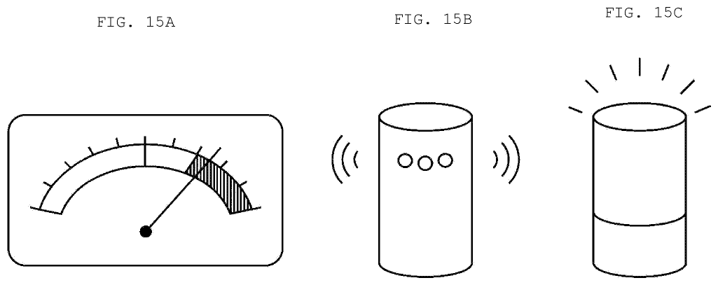

Next, FIG. 5 is a diagram illustrating the processing of the individual psychology diagnosis unit 21. Based on the behavior xᵢ (ε) of the individual i, the constraint conditions, and the like, the individual psychology diagnosis unit 21 calculates a utility value function uᵢ(x, ε) (solid line) and a socially normative value function nᵢ(x, ε) (dashed line), and the intersection of the two is estimated as the value vᵢ (ε) for the behavior xᵢ (ε) of the individual i. Hereinafter, the utility value function uᵢ(x, ε) will be referred to as a utility function uᵢ (x, ε), and the socially normative value function nᵢ(x, ε) will be referred to as a normative function nᵢ(x, ε).

As shown in the drawing, it is assumed that the utility function uᵢ(x, ε) has an upward slope with respect to the environment ε, and conversely the normative function nᵢ(x, ε) exhibits a downward slope as a mandatory constraint. In this case, at the intersection of the two, the behavior xᵢ (ε) that balances the selfish utility function uᵢ(x, ε) and the altruistic normative function nᵢ (x, ε) will be: selected.

That is, it is possible to obtain the equation Uᵢ(xᵢ, ε)=nᵢ(xᵢ, ε) at the intersection of the two functions, approximately a linear equation consisting of four variables (two slopes and two intercepts) for the environment ε.

For these four variables, if the four variables are obtained by solving the linear equations, with two constants determined from a linear approximation function of the behavior xᵢ (ε) of the individual i measured with respect to the environment ε, and two constraints, for example, the upper li.mi.t of the utility function and the lower limit of the normative function, it is possible to obtain linear approximation functions representing the utility function uᵢ(x, ε) and the normative function nᵢ(x, ε), respectively.

Next, FIG. 6 is a diagram illustrating the processing of the intervention unit 22. The normative value nᵢ (xᵢ, cᵢ, r, ε) (=nᵢ(xᵢ, ε)) or fairness fᵢ(xᵢ, cᵢ, r, ε) of the individual i with respect to the environment ε shown by the solid line in the drawing can. change in time series.

Here, cᵢ is the characteristic of the individual i, and r specifies the operation rule under the current institution 13.

Fairness fᵢ(xᵢ, cᵢ, r, ε) is expressed using vᵢ(ε)/cᵢ, for example, as an operation rule r, following the fairness theory by Adams.

The average of others as fₐ (=1/(n-1)·Σvⱼ(ε)/cⱼ) is calculated, and it can be determined to be fair if fᵢ=fₐ, to be selfish if fᵢ>fₐ, and to be altruistic if fᵢ<fₐ.

From this point of view, the normative value nᵢ(xᵢ, cᵢ, r, ε) or the fairness fᵢ(xᵢ, cᵢ, r, ε) of the individual i is compared with a normative standard nₛ(r, ε)±σₙ or a fairness standard fₛ (r, ε)±σ_{f} determined from the current operation rule r and the environment ε indicated by two dashed lines centered on the dashed-dotted line. Here, nₛ(r, ε) is the reference value of the normative standard, fₛ(r, ε) is the reference value of the fairness standard, and σₙ and σ_{f} are the tolerances.

The reference value fₛ (r, ε) of the fairness standard may be set to the average fₐ of others. In this case, the reference value nₛ (r, ε) of the normative standard is equal to the normative value nᵢ(xᵢ, ε) of the individual i when the fairness fᵢ of the individual i=the reference value fₛ of the fairness standard holds. The reference values fₛ and nₛ are updated based on mutual comparison with others j (≠i) in the interpersonal interaction 12.

A shaded area 161 shown in the drawing indicates that the normative value nᵢ(xᵢ, cᵢ, r, ε) or the fairness fᵢ(xᵢ, cᵢ, r, ε) of the individual i deviates from the normative standard nₛ (r, ε)±σₙ or the fairness standard fₛ(r, ε)±σ_{f}.

When fairness is used as the operation rule r, unfairness occurs in the shaded area 161. Thus, if the normative value nᵢ(xᵢ, cᵢ, r, ε) or fairness fᵢ(xᵢ, cᵢ, r, ε) of the individual i deviates from standards, the IoH device 100 can prompt the individual i to behave in a normative or fair manner by generating intervention data (for example, image data to be displayed on a display as the user I/F 150) with the intervention unit 22 and using the user I/F 150. By receiving such an intervention, the individual i can be expected to review his/her own behavior and make it a habit by taking responsibility for himself/herself.

Social norms are based on mutual recognition between individuals, and intervention in the individual behavior 11 by the intervention unit 22 can be substantially regarded as an intervention in the interpersonal interaction 12 by the intervention unit 22. In addition, the intervention unit 22 can acquire the tendency of the behavior formation of the individual i from. the consensus building unit 26 of the slow system 32 and use the tendency to generate intervention data

The tolerance σₙ or σ_{f} is determined in consideration of the variation in behaviors of a plurality of individuals. For example, if a particular individual i's normative value nᵢ (xᵢ, cᵢ, r, ε) or fairness fᵢ(xᵢ, cᵢ, r, ε) frequently deviates from the normative standard nₛ(r, ε)±σₙ or fairness standard fₛ(r, ε)±σᵢ toward selfishness, then the individual i may be considered a free rider and given a stronger warning rather than intervention. In addition, after each individual i obtains consent when joining the fast system 31, the individual i may be excluded from the fast system 31 if the behavior of the individual i does not change in response to repeated warnings.

In addition, if the number of individuals i who deviate from the standard exceeds a predetermined threshold, it is determined that the current operation rule r or institution 13 should be improved, and the second circulation loop by the slow system 32 may be started.

<Modification of Fast System 31>

Another method of obtaining' the utility function uᵢ(x, ε) and the normative function nᵢ(x, ε) in the fast system 31 may estimate a utility value score and a normative value score by building a psychological model with machine learning of the data of the behavior xᵢ(ε) of the individual i.

Also, by performing a meta-analysis of psychological intervention experiments by machine learning, the psychological model obtained therefrom may be reflected in the intervention of the intervention unit 22 for the individual i. Furthermore, if machJne learning is applied to the promotion results of normative behaviors for the content of intervention, more effective intervention can be carried out.

In addition, when performing a calculation with a large load such as machine learning, the calculation is performed in the server 200 instead of performing a calculation in the IoH device 100, and the model obtained as the calculation result may be transmitted to the IoH device 100 via the network 170.

<Application Example of Fast System 31>

The fast system 31 can be applied, for example, to a distributed cooperative system that operates by regarding renewable energy as a shared resource. In this application example, the environment ε can be considered as temperature, the behavior xᵢ(ε) of the individual i as power consumption, the utility function uᵢ(xᵢ, ε) as the comfort level, and the normative function nᵢ(xᵢ, ε) as power saving awareness. Then, the value vᵢ (ε) can be obtained by selecting the behavior xᵢ (ε) that balances comfort and power saving.

The characteristics cᵢ of the individual i depend on, for example, physicality, residence, number of cohabitants, number rooms, building structure, air conditioning equipment, and the like. For example, if the individual i is regarded as a household, the characteristric cᵢ may simply be the number of household members, but the number of household members may also be adjusted by weighting children, elderly persons, disabled persons, and the like.

In this application example, power consumption with respect to temperature corresponds to the first data related to individual behavior. In addition, comfort level, power saving awareness, value, fairness based on mutual comparison with others, and the like correspond to the second data related to interpersonal interaction. In addition, statistical data such as total, average, and variance obtained from each individual's first data and second data, as well as total renewable energy power generation and costs related to the operation, correspond to the third data related to the current institution.

The fast system 31 intervenes in social norms based on the individual behavior and the interpersonal interaction 12 by encouraging the individual i to behave in a normative or fair manner, and as a result, the first data or the second data can be updated.

In this application example, each IoH device 100 collects temperature as the environment ε and power consumption as the behavior xᵢ(ε) of the individual i. In addition, operation data such as the state of renewable energy as a shared resource and the operating state of facilities are transmitted to each IoH device 100, and the distributed cooperative operation is performed by the interpersonal interaction 12. Data relating to these individual behaviors, interpersonal interactions, environments, and operations are transmitted and stored in the server 200 or the database 270 that constitutes the slow system 32, and are used by the group state diagnosis unit 23 to diagnose the group. Thus, the operation incorporating the social norms of the fast system 31 and the continuous consensus building of the slow system 32 are interlocked.

<Configuration Example of Slow System 32>

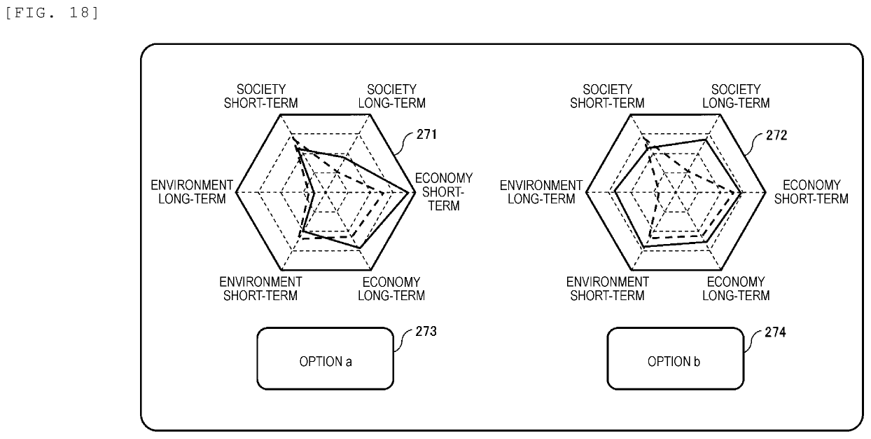

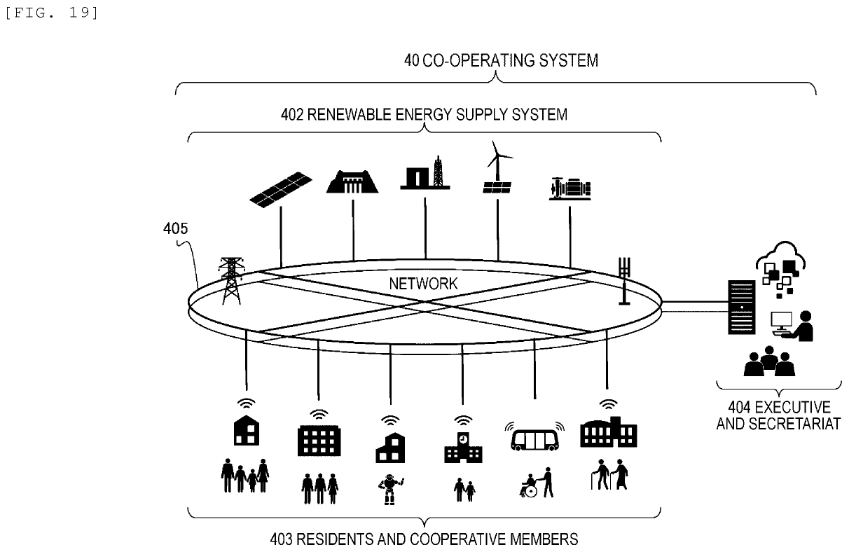

Next, FIG. 7 shows a configuration. example of the slow system 32. The slow system 32 is composed of the server 200 and the database 270 to which each IoH device 100 is connected via the network 170.

The server 200 includes a processor 210 such as a CPU, a memory 220 such as a DRAM, a storage 240 such as an HDD or an SSD, a user I/F 250 such as a touch panel and a display, and a network I/F 260 such as a communication module.

In the server 200, an operation data collection program 231, a group state diagnosis program 232, a prognosis prediction generation program 233, an option generation program 234, a consensus building program 235, and an institution update program 236 which are stored in the storage 240 are read into the memory 220 and executed by the processor 210 to implement the collection unit 23a, the group state diagnosis unit 23, the prognosis prediction. unit 24, the option generation unit 25, and the consensus building unit 26, and the update unit 26a belonging to the slow system 32 of the functional blocks constituting the information system 20.

If the fast system 31 composed of the plurality of IoH devices 100 is a responsive distributed cooperative system of shared resources, as in the example above, the slow system 32 composed of the server 200 functions as a deliberative centralized management system for the plurality of IoH devices 100. The server 200 as a centralized management system updates the institution 13 according to relatively large changes in economic situations, environmental conditions, and the like.

In the server 200, under the current institution 13, the collection unit 23a collects operation data from each IoH device 100, the group state diagnosis unit 23 diagnoses the state of the group based on the operation data, and the prognosis prediction unit 24 prepares scenarios of various prognosis predictions, and the option generation unit 25 generates options for the future institution in the social group based on these scenarios. Furthermore, the consensus building unit 26 presents options to each individual to support group consensus building, and the update unit 26a updates the current institution 13 based on the group consensus. The updated institution 13 is notjfied to each IoH device 100, thereby operating shared. resources under the updated institution 13.

Next, the processing of the collection unit. 23a, the group state diagnosis unit 23, the prognosis prediction unit 24, the option generation unit 25, and the consensus building unit 26 belonging to the slow system 32 will be described in detail.

FIG. 6 is a diagram illustrating the processing of the collection unit 23a and the group state diagnosis unit 23. The collection unit 23a collects operation data from each IoH device 100 and performs statistical processing to obtain time-series data of the group state s as shown in the drawing. The group state diagnosis unit 23 diagnoses whether the group is in an equilibrium state (stationary state) or in a non-equilibrium state by obtaining the autocovariance or autocorrelation of the time-series data of the group state s. When the group state diagnosis unit 23 diagnoses that the group state a is in non-equilibrium, it is necessary to stabilize the state by updating the current institution 13, and thus, the operations after the prognosis prediction unit 24 are started.

Next, FIG. 9 is a diagram illustrating the processing of the prognosis prediction unit 24. As shown in the drawing, the prognosis prediction unit 24 simulates various combinations institutions 13 (operation rules for individuals, operation conditions such as facilities and operations), and trial-calculates the value of the group for each option. After clarifying the association network of various value index groups related to society, environment, and economy, by summarizing them, the social value, environmental value, and economdc value as three representative indices are obtained.

Note that, instead of social value, environmental value, and economic value, cultural values such as history, traditon, customs, climate, landscape, and the like may be selected as representative indices. Further, the happiness level, QoL (Quality of Life), and the like, which are obtained by comprehensively quantifying the various values described above, may be used as representative indices. The number of representative indices is not limited to three and can be arbitrarily increased or decreased. Note that, if the number of representative indices is increased too much, there is a concern that the understanding of the options will diverge, and if the number is reduced too much, there is a concern that the trade-offs and dilemmas between indices will become difficult to understand

Next, FIG. 10 is a diagram illustrating the processing of the option generation unit 25. The three representative indices for various options obtained by the prognosis prediction unit. 24 can be plotted on a triangular graph as shown in the drawing.

The point p in the drawing indicates the position of value with respect to the current group state s. A shaded triangle indicates a range in which the group state s is predicted to be stable based on the simulation by the prognosis prediction unit 24 among many options. The option generation unit 25 generates four points, i.e., the three points a, b, and c within the stable range (shaded triangle) and the inner center point d of the stable range, as options. The option generation unit 25 may acquire intervention data for the individual behavior 11 or the interpersonal interaction 12 from the intervention unit 22, refer to the intervention data to determine the tendency of each individual i or group, for example, which is emphasized among the social value, environmental value, or economic value, and select the options to be presented based on that determination.

In the drawing, the three representative indices are plotted and visualized on the triangular graph, but Sankey diagram, a three-dimensional graph, or the like may be used instead of the triangular graph. Note that, when using a three-dimensional graph, the plots of various options appear to overlap in the depth direction, which requires study.

Next, FIG. 11 is a diagram illustrating the processing of the consensus building unit 26 and the update unit 26a. The consensus building unil 26 determines the order of representative options a, b, c, and d by making each individual i use the user I/F 150 of the IoH device 100 to vote or select. In addition to the malority rule and the Boulder rule shown in the drawing, methods for determining the order include Condorcet's method, Young's method, AHP (Analytic Hierarchy Process) method, and the like.

Alternatively, after trial calculation of the value vᵢ(ε) of each individual i for the options a, b, c, and d, the order may be calculated based on social welfare standards. The social welfare standards include utilitarian standards, maximin standards, and leximin standards.

From the perspective of social welfare, there is a concern that majority rules and utilitarian standards may not respect minorities who receive only low value. For example, the boulder rule has the advantage that the option that results in the all-out loser of the binary choice is not chosen, and the leximin standards do not choose the option evaluated as the lowest. value. Therefore, by presenting each individual i with options that include both an order based on voting or selection and order based on social welfare standards, each individual i can be given the perspective of respecting diversity and minorities, which can be reflected in the consensus building.

In addition, in order to persuade individuals who assert selfish selfishness against respect for minority opinions, it is possible to present the options to the relevant person after re-evaluating them in light of some universal value. Moreover, if consent is obtained from all the subscribers when joining the slow system 32, it is possible to take a method such as excluding the relevant person from the consensus building process.

The consensus building unit 26 feeds back the results of the order of representative options determined using a Predetermined method to the IoH device 100 of each individual i (for example, by displaying them on the user I/F 150) to encourage a vote or selection for one of the representative options. At this time, a compromise plan may be presented to each individual by estimating an option that is likely to lead to a consensus by all members or a route that will likely lead to a consensus.

In addition, the consensus building unit 26 may refer to the intervention data for the individual behavior 11 or the interpersonal interaction 12 by the intervention unit 22 to grasp each individual i or the group's tendency, for example, being selfish or economically oriented, or being altruistic or socially oriented and environmentally oriented, thereby searching for a compromise plan that is easy to reach a consensus. In order to avoid the continuation of the unstable state of the group, it is possible exceptjonally to select a provisional option before the consensus building unit 26 reaches a consensus.

In this way, by repeatedly voting or selecting representative options and giving feedback, after an option that finally reached a consensus is obtained, the update unit 26a updates the institution 13 and notifies each individual i's IoH device 100 of the new institution 13. As a result, the institution 13 updated based on the group consensus building is reflected in the individual behavior 11 and the interpersonal interaction 12.

As a method of obtaining a group consensus, as described above, in addition to the method of repeating voting or selection and feedback on representative options, for example, a method in which opinions on options are collected from each individual i and representative opinions are displayed, a method of grouping and displaying the opinions of all members by the KJ method or language analysis, or the like may be used.

In the above description, in the slow system 32, the prognosis prediction unit 24 and subsequent units take the opportunity that the Group state s becomes non-equilibrium by the group state diagnosis unit 23 to start processing. As another operation of the slow system 32, before the group state s reaches a completely non-equilibrium state by the group state diagnosis unit 23, the group state diagnosis unit 23 may take the opportunity that such a sign appears to notify the intervention unit 22 of the fact. As a result, the intervention of the slow system 32 in the fast system 31 can be strengthened, and the group state s can be prevented from becoming unstable.

Also, in practice, by operating the slow system 32 in parallel with the fast system 31, it is possible to calculate the value index based on prognosis prediction and extract representative options while receiving operation data from the fast system 31.

As a result, it is Possible to evaluate various institutions and operation rules r in advance, and when it becomes necessary to update them, the process is possible to quickly proceed to consensus building.

As a modification of the slow system 32, in order to improve followability to relatively small changes rather than large changes in economic situations and environmental conditions, the consensus building unit 26 may omit consensus building and select options based on a predetermined policy, and the update unit 26a may update the institution 13. This policy is assumed to have been agreed upon by the subscribers when subscribing to the slow system 32.

<Application Example of Slow System 32>

As described above, when the fast system 31 is applied to a distributed cooperative system that operates by regardng renewable energy as a shared resource, the slow system 32 can be applied to a centralized management system that updates the operation rule r of a distributed cooperative system (fast system 31) in response to relatively large changes of economic situations such as costs and environmental conditions such as the amount of solar radiation and water.

In this case, the group state s corresponds to the total power consumption of the entire population or the total power consumption normalized by the temperature ε, the social value corresponds to the composite index of the local economic circulation rate (regional activity level) and the power saving comfort level of the residents, the environmental value corresponds to the renewable energy self-sufficiency rate, and the economic value corresponds to the reciprocal of the energy cost.

In addition, the representative options a, b, and c correspond to social, environmental, and economic emphasis within the stable range of prognosis prediction, and the option d is moderate. From among them, a new institution, that is, the operation rule r is selected by building a consensus among residents.

For example, if a description is made using the triangular graph shown in FIG. 10, the option c emphasizing economic value as the operation rule r is intended to reduce electricitycharges as much as possible by using energy other than renewable energy. Thus, the normative standard nₛ(r, ε) or the fairness standard fₛ (r, ε) for power saving is somewhat looser than the option p, which represents the current group state s.

The option b, which emphasizes environmental value, is intended to increase the renewable energy self-sufficiency rate as much as possible. Therefore, if the option b, which. emphasizes the environment, is selected, electricity charges will rise compared to the option c, which emphasizes economic value. in addition, the normative standard nₛ (r, ε) or the fairness standard fₛ(r, ε) as the operation rule r encourages power saving more strictly than. the option p representing the current group state s.

The option a, which emphasizes social value, is intended to balance the improvement of the regional activity level through renewable energy self-sufficiency and the comfort level of the residents. Therefore, although the electricity charges will be higher than the option c, which emphasizes economic value, the standard for power saving will be looser, like the option c.

The representative options a, b, c, and d as the operation rule r are generated by the option generation unit 25 through the diagnosis of the group state and the prognosis prediction based on the firs data, the second data, and the third data collected by the collection unit 23a, and the consensus building unit 26 supports consensus building as a social group for any of the options a, b, c, and d.

Then, of the options a, b, c, and d, the one finally agreed upon becomes the operation rule r and is newly applied to the fast system 31. As a result, the operation status as a social group changes, and the third data is updated. Specifically, for example, in the case of the occurrence of changes in weather or disasters related to renewable energy power generation, maintenance or failure of power generation equipment, revision of electricity charges for non-renewable energy, and the like, the operation rule r is updated and the third data changes significantly.

<Display Example on User I/F 150 of IoH Device 100>

Next, the description will be made with reference to FIGS. 12 to 14 on the screen display when a display is adopted as the user I/F 150 of the IoH device 100 when the fast system 31 is applied to a distributed cooperative system and the slow system 32 is applied to a centralized management system for updating the operation rule r of the distributed cooperative system.

FIG. 12 shows a display example of a screen displayed on the user I/F 150 of the IoH device 100 when the intervention unit 22 of the distributed cooperative system (fast system 31) intervenes in the individual behavior 11.

In the drawing, a display area 151 on the left side of the screen displays a solid curve 151a that indicates the transition of the power consumption xᵢ(ε) of the individual i, and a dashed curve 151b that indicates the transition of the total amount of renewable energy generated. A display area 152 on the right side of the screen displays a dashed straight line 152a that indicates the tolerance of the fairness standard fₛ(r) normalized by the temperature c, and a solid curve 152b that indicates the transition of the fairness fᵢ(vᵢ, cᵢ, r) of the individual .i normalized by the temperature ε. The fairness fᵢ of the individual and standard fₛ represent fair if fᵢ=fₛ, selfish if fᵢ>fₛ, and altruistic if fᵢ<fₛ.

By looking at this screen display, each individual i can recognize that the transition of his/her own power consumption xᵢ (ε) deviates from the transition of the total power generation and that his/her own fairness fᵢ deviates from the range of the fairness standard fₛ(r) based on mutual comparison with others j. Therefore, each individual i can be encouraged to take normative and fair power consumption behavior.

The normative value nᵢ(xᵢ, cᵢ, r) may be displayed instead of the fairness fᵢ(vᵢ, cᵢ, r) of each individual In addition, on the screen for intervening in the individual behavior 11, individuals with altruistic fairness fᵢ may be displayed as power-saving rankings, and evaluations such as “like” and “disappointing” from others may be displayed based on the fact that social norms are enabled through mutual recogniton.

FIG. 13 shows a display example of a screen displayed on the user I/F 150 of the IoH device 100 when the consensus building unit 26 of the centralized management system (slow system 3) supports the consensus building of the social group on the options.

In the drawing, a triangular graph 153 with social value, economic value, and environmental value as vertices is displayed on the left side of the screen, and on the triangular graph 153, the values corresponding to the option p representing the current group state s and the representative options a, b, c, and d are plotted. In the area 154 on the right side of the screen, the order of the previous selections of all members of the group for the representative options a, b, c, and d, the order based on social welfare standards, the order based on social welfare standards, and the result of the previous selection and the contents of the current selection of the individual i are shown.

In the processing by the consensus building unit 26, the individual i learns by comparing his/her previous selection results with selection results of all members and social welfare standards, and consensus building and social welfare are promoted for the will of the individual i. Then, the individual i operates a puil-down menu button 155 to select the order that he/she considers tolerable and that all members will reach a consensus, as the current decision. As each individual i performs the same operation, a group consensus is gradually built.

Instead of the triangular graph 153, a Sankey diagram or a three-dimensional graph may be displayed, or numerical values or relative values of social value, economic value, and environmental value may be displayed. Also, instead of showing several orders of options, the majority opinion or minority opinion for each option may be displayed, or a compromise plan that is likely to reach a consensus may be displayed as a recommendation.

FIG. 14 shows a display example of a screen displayed on the user I/F 150 of the IoH device 100 after a consensus of the social group is obtained by the consensus building unit 26 of the centralised management system. (slow system 32).

In the drawing, a triangular graph 156 with social value, economic value, and environmental value as vertices is displayed on the left side of the screen, and on the triangular graph 156, the agreed option d is highlighted and displayed larger than the other options.

In the upper right area 157 of the screen, the order based on various selection rules and social welfare standards for representative options a, b, c, and d is displayed. In the example of the drawing, in any of the majority rule, the Boulder rule, the utilitarian standard, and the leximin standard, the option d is at the top, or has a semi-transitive relationship or an indiscriminate relationship with the top option, which indicates that the social group agreed to the option d.

In the lower right area 158 of the screen, the goal of the operation rule as the new institution 13 updated by the update unit 26a is displayed. In the example of the drawing, the electricity charge ratio for the case of 0% renewable energy corresponding to the economic value, the renewable energy self-sufficiency rate corresponding to the environmental value, the regional activity level (local economic circulation rate) corresponding to the social value, and the (ratio of) comfort level at the time of the renewable energy shortage (compared to when there is sufficient supply) are shown.

Thus, after the institution 13, i.e., the operation rules are updated, the distributed cooperative system supplies renewable energy and other energies each individual i, while an intervention on social norms or fairness is performed on the power consumption behavior of the individual i and operation according to the rules as a whole social group is performed.

Next, FIGS. 15A-15C show examples of the user I/F 150 other than the display. FIG. 15A of the same drawing shows an example of a level meter as the user I/F 150. In the level meter, for example, the median value of the meter can be defined as the fairness standard and the shaded portion as the selfish deviation region to indicate the fairness value of the individual i with respect to the fairness standard.

FIG. 15B of the same drawing shows an example of a smart speaker as the user I/F 150. In the smart speaker, for eample, the pitch, intensity, chord, melody, and the like of the output sound can represent the fairness of the individual i with respect to the fairness standard. An alarm sound, warning voice, or the like may be output only when the fairness of the individual i deviates from the fairness standard.

FIG. 15C of the same drawing shows an example of a smart light as the user I/F 150. In the smart light, for example, the fairness of the individual i with respect to the fairness standard can be represented by the color, intensity, change, or the like of the emitted light. Blinking or flashing light may be output only when the fairness of the individual i deviates from the fairness standard.

The above-mentioned level meter, smart speaker, and smart light can also serve as power sensors and remote controllers for home appliances and lighting, sensors for environmental temperature, humidity, and air cleanliness, and sensors for human body temperature, pulse, and physical activity.

Although illustration is omitted, a vibrator or a diffuser is adopted as the user I/F 150, and the fairness of the individual i with respect to the fairness standard may be represented according to the intensity and period of vibration and the type of fragrance.

As described above, by using the user I/F 150 to make each individual i aware of his/her own fairness and deviation from the fairness standard, it is possible to encourage the individual i to take normative behavior. This makes it possible to operate the fast system 31 stably.

<Display Example on User I/F 250 of Server 200>

Next, the screen display on the user I/F 250 of the server 200 when the fast system 31 is applied to a distributed cooperative system and the slow system 32 is applied to a centralized management system for updating the operation rule r of the distributed cooperative system will be described.

FIG. 16 shows a display example of a screen displayed on the user I/F 250 of the server 200.

In the drawing, in the upper left area 251 of the screen, transition to date (thick solid line) and future forecast (thick dashed line) of the supply amount of renewable energy in the distributed cooperative system as the fast system 31, as well as transition to date (thin solid line) and future forecast (thin dashed line) of the amount of demand (amount of consumption) are displayed. In the example in the drawing, as an example, the case where the demand forecast exceeds the supply forecast is displayed.

In the lower left area 252 of the screen, the transition to date (solid line) and the future forecast (dotted line) of the degree of deviation from the fairness standard of the entire group in the distributed cooperative system as the fast system 31 are displayed. The degree of deviation in the example of the drawing is the sum of the absolute values of the degree of deviation for each individual i, and the larger the degree of devation, the more unstable the state of the group.

In the upper right area 253 of the screen, the history to date of consensus building by the centralized management system as the slow system 32 is displayed on a triangular graph with social value, environmental value, and economic value as vertices. The open circles represent the currently agreed options, that is, the positions of the current institution 13. The example in the drawing shows the case where the group's consensus is currently approaching the inner center of the triangle after shifting from emphasizing the economy to emphasizing the environment and then emphasizing the society.

In the lower right area 254 of the screen, transition to date (solid. line) and future forecasts (dotted line) of the relative index values of social, environmental, and economic values in the distributed cooperative system as the fast system 31 operated under the current institution 13 are displayed. The dashed-dotted line corresponds to the index value of the inner center d (FIG. 10) in the triangular graph. Since the three values of the society, the environment, and the economy are representative indices of various index groups, they cannot be generalized. Here, the three values are almost unchanged since the quality of life and the energy self-sufficiency rate are not changed while the power supply sufficiently exceeds the demand thereof, but if the demand forecast exceeds the supply, forecast, the social value and environmental value will decrease, and the economic value will increase relatively.

The server 200 continuously executes the second circulation loop consisting of operation data collection, group state diagnosis, prognosis prediction generation, option generation, consensus building support, and institution update.

With the user I/F 250, the state of each. process in the second circulation loop can be monitored from a bird's-eye view. In addition, in the user I/F 250, for example, when the demand forecast for power exceeds the supply forecast, when a predetermined emergency operation rule is applied due to a disaster or failure, and the like, or when switched to the operation mode for maintenance, the administrator of the centralized management system as the slow system 32 can input a predetermined instruction operation.

In addition, in the example of FIG. 16, for they sake of clarity, an abnormal situation in which the demand forecast exceeds the supply forecast is shown. Normally, however, when the supply amount exceeds the demand amount, surplus power is used for power storage, heat storage, pumping, or the like, and the stored power compensates for the shortage of power when the supply amount decreases, thereby the supply and demand balance is maintained.

Next, FIG. 17 shows another display example of the screen displayed on the user I/F 150 of the IoH device 100 when the consensus building support is provided by the consensus building unit 26 of the centralized management system (slow system 32).

In the drawing, on the left side of the screen, three vertices of social value, environmental value, and economic value and the inter center on the triangular graph respectively correspond to the R, G, B, and N on the color triangle in a chromaticity diagram 261. Also, on the right side of the screen, a color triangle 262 consisting of social value (corresponding to R in the color triangle), environmental value (corresponding to B in the same color triangle), and economic value (corresponding to B in the same color triangle) is displayed, and the current group state p and representative options a and b are plotted thereon. Further, on the right side of the screen, operation buttons 263 and 264 corresponding to the options a and b are provided. The display colors of the operation buttons 263 and 264 are the same as the colors of the points of the options a and b in the color triangle 262. In the drawing, the operation button 263 is displayed in orange and the operation button 264 is displayed in yellowish green.

Each individual i who is a user of the IoH device 100 can intuitively understand the positions of the options a and b in the color triangle 262 from the display colors of the operation buttons 263 and 264. Each individual i can select the option a or the option b by operating the operation button 263 or the operation button 264.

For example, the brightness of the options a and b plotted on the color triangle 262 may be changed according to their relative amount of support. By doing so, it becomes possible for each individual i to recognize which of the options a and b is supported by the majority.

Next, FIG. 18 shows stjll another display example of the screen displayed on the user I/F 150 of the IoH device 100 when the consensus building support is provided by the consensus building unit 26 of the centralized management system (slow system 32).

In the drawing, a hexagonal radar chart in which social value, environmental value, and economic value are respectively divided into short-term value and long-term value is used instead of a triangular graph with social value, environmental value, and economic value as vertices. The radar chart 271 on the left side of the screen shows the value of the option a (solid line) and the value of the current state p (dotted line), and the radar chart 272 on the right side of the screen shows the value of the option b (solid line) and the value of the current state p (dotted line).

In addition, operation buttons 273 and 274 corresponding to the options a and b are provided on the lower side of the screen. Each individual i who is a user of the IoH device 100 can understand the characteristics of the options a and b from the shapes of the radar charts 271 and 272. Each individual i can select the option a or the option b by operating the operation button 273 or the operation button 274.

For example, the thickness of the solid lines of the radar charts 271 and 272 and the saturation and brightness of the operation buttons 273 and 274 may be changed according to the relative number of instructions. By doing so, it becomes possible for each individual i to recognize which of the options a and b is supported by the majority.

<Application Example of Co-operating System 1>

Next, FIG. 19 shows a configuration example of a co-operating system 401 in a local community, a cooperative, and the like, to which the co-operating system 1 according to the present embodiment is applied.

The co-operating system 401 includes a renewable energy supply system 402, the IoH device 100 (not shown), and residents and cooperative members who consume renewable energy and an executive and secretariat 404 of the renewable energy supply system 402 including the server 200 (not shown) are connected to each other via a network 405 consisting of a power transmission and distribution network and a communication network.

The energy supply sources of the renewable energy supply system 402 are sunlight, hydraulic power, biomass, wind power, small hydraulic power, and the like, and constitute a microgrid.

Residents and cooperative members 403 are households, companies such as offices and factories, public facilities such as schools and public halls, and transportation means such as buses and cars, participate in the co-operating system 401, and consume renewable energy under the autonomous operation rules of the fast system.

The executive and secretariat 404 monitor the renewable energy supply system 402 using the server 200, and the executive and secretariat 404 respond to maintenance and emergencies.

In the co-operating system 401, the IoH devices 100 used by the residents and cooperative members 403 implement a distributed cooperative system as the fast system 31 that operates renewable energy, which is a shared resource. Also, the server 200 provided in the executive and secretariat 404 implements a centralized management system as the slow system 32 that updates the operation rules of the distributed cooperative system in response to economic situations and environmental changes.

<Other Application Examples of Co-operating System 1>

In the above description, the fast system 31 is applied to a distributed system and the slow system 32 is applied to a centralized system, but application. examples of the fast system 31 and the slow system 32 are not limited thereto.

For example, it is possible to configure both the fast system 31 and the slow system 32 as a centralized system by assigning the IoH device 100 or IoT device to the sensing function of the behavior data and operation data. Conversely, by transferring some of the functions of the slow system 32, such as preprocessing of behavior data or operation data, to the fast system 31, it is also possible to configure from a centralized system to a distributed system.

System configuration to be adopted may be designed according to the scale, processing speed, flexibility, robustness, scalabljtv, and the like required for the co-operating system 1.

Although it is possible to construct the fast system 31 and the slow system 32 independently without cooperating with each other, as in the present embodiment, since there is a mutual relationship between the two, it is useful to have both cooperate, as in the co-operating system 1 of the present embodiment. This is obvious from the fact that the social system 10 consists of the first hierarchy 11 related to individual behaviors, the second hierarchy 12 related to interpersonal interactions, and the third hierarchy 13 related to institution of groups, and that both operation (administration) and consensus building (politics) are necessary for the social system 10.

In the above description, renewable energy was mentioned as a shared resource, but there are other shared resources such as farmland, irrigation, forestry, fishing, and resources. It also covers mobility such as car sharing and on-demand buses, and the sharing economy related to labor such as coworking, work sharing, and cooperatives. It can also be extended to fair and equitable economic transactions, allocation or carbon dioxide and waste emissions, and so on Social issues such as disparities, inequalities, resources, and the environment involve not only economic utility but also social norms, ethics, and moralities, and the co-operating system 1 of the present embodiment can be widely applied to resolve social issues.

<Summary>

According to the social system theory of sociologist Niklas Luhmann, a social system is an autopoiesis (self-producing) system consisting of a cyclical network of communication between individuals. In the co-operating system 1 of the present embodiment, a cyclical communication network between the social system 10 and the information system 20 is constructed.

According to philosopher Joseph Heath's social norm theory, social norms (rules, ethics, and morality) are enabled by a structure of mutual expectations and approval between individuals based on the human propensity to conform to imitation. In the co-operating system 1 of the present embodiment, the information system 20 normatively intervenes in interactions between individuals in the social system 10.

According to economist Masahiko Aoki's comparative institutional analysis, a social system is a self-sustaining system consisting of an inddvidual's beliefs about a group, the individual's strategic behavior based on that belief, the balance of the group that is generated by gathering these beliefs, and a symbolized summary expression, that is, an institution, and a cyclical loop of coordinated individual beliefs. In the co-operating system 1 of the present embodiment, an information system intervenes between the circulation loops of individuals and groups.

According to system engineer Takehiro Inohara's theory of consensus building, consensus building should be understood not as social integration based on majority voting or the greatest common divisor, but as social editing that accepts, relates, and harmonizes diversity and differences. In the co-operating system 1 of the present embodiment, the information system supports the social editing function in consensus building.

From the point of view of system theory, life tissue forms a hierarchical structure by uhe emergence of order from the lower layer to the upper layer, such as cell→tissue→organ→organism→group→ecosystem. According to the theory of double inheritance, humans are the product of the interplay of biological and cultural evolution, seen as a co-evolution of genomes and memes. For these reasons, the co-operating system 1 of the present embodiment has a hierarchical structure.

According to behavioral economist Daniel Kahneman, uhe human thought system consists of a dual process: an intuitive, automatic fast system and a conscious, deliberative slow system. In response to this, philosopher Joseph Heath advocated slow politics based on reason and deliberation in politics and economics against fast politics based on intuition and emotion. Therefore, the co-operating system 1 of the present embodiment constitutes a double hierarchy of an operation system based on the fast system 31 and a consensus building system based on the slow system 32.

According to the co-operating system 1 of the present embodiment, automatic operation is performed by the first circulation loop by the fast system 31, and deliberative consensus building is performed by the second circulation loop by the slow system 32. Then, the fast system 31 follows daily fluctuations in the economy and the environment and operates in a responsive manner, and the slow system 32 continuously builds consensus on relatively large changes. By operating both in parallel, it is possible to adapt to various situations.

In addition, according to the co-operating system 1 of the present embodiment, the fast system 31 intervenes in the individual behavior 11 or the interpersonal interaction 12 in light of the current institution 13 that reflects the prior consensus, thereby making it possible to operate in consideration of social norms as well as economic utility. In addition, by presenting institution options based on the group state under the current institution 13, the slow system 32 makes it possible to build consensus not only on the economy but also on various social values such as the environment and culture.

The co-operating system 1 includes both the meaning of cooperation between the sociai system 10 and the information system 20 and cooperation between the fast system 31 and the slow system 32. These two types of cooperation enable the social system to operate normatively and to continue without failure.

Although the embodiments and modifications according to the present invention have been described above, the present invention is not limited to the above-described examples of the embodiments and includes various modifications. For example, the above-described exemplary embodiments are described in detail to facilitate understanding of the present invention, and the present invention is not limited to those having all uhe configurations described herein. Also, part of uhe configuration of one example of an embodiment can be replaced with the configuration of another example. Also, it is possible to add another example configuration to one example configuration of a certain embodiment. In addition, the addition of other configurations, deletion, or replacement can be made with respect to part of the configuration of the example of each embodiment. Further, each of the above configurations, functions, processing units, processing means, and the like may be implemented by hardware, for example, by designing a part or all of them. using an integrated circuit. In addition, the control lines and information lines in the drawing indicate what is considered necessary for the description, and do not necessarily indicate all of them. It may be considered that almost all configurations are interconnected.

In addition, the configuration of the co-operating system described above can be classified into more components according to the processing content. Also, one component can be further classified to perform more processing.

### REFERENCE SIGNS LIST

1 . . . co-operating system, 10 . . . social system, 11 . . . first hierarchy (individual behavior), 12 . . . second hierarchy (interpersonal interaction), 13 . . . third hierarchy (institution), 20 . . . information system, 21 . . . individual psychology diagnosis unit, 21a. . . collection unit, 22 . . . intervention unit, 23 . . . group state diagnosis unit, 23a. . . collection unit, 24 . . . prognosis prediction unit, 25 . . . option generation unit, 26 . . . consensus building unit, 26a. . . update unit, 31 . . . fast system, 32 . . . slow system, 100 . . . IoH device, 110 . . . processor, 120 . . . memory, 131 . . . behavior data acquisition program, 132 . . . individual psychology diagnosis program, 133 . . . intervention data generation program, 140 . . . storage, 150 . . . user I/F, 170 . . . network, 200 . . . server, 210 . . . processor, 220 . . . memory, 231 . . . operation data collection program, 232 . . . group state diagnosis program, 233 . . . prognosis prediction generation. program, 234 . . . option generation program, 235 . . . consensus building program, 236 . . . institution update program, 240 . . . storage, 250 . . . user I/F, 270 . . . database, 401 . . . co-operating system, 402 . . . renewable energy supply system, 403 . . . residents and cooperative members, 404 . . . executive and secretariat, 405 . . . network

1. An information system comprising:
a first system including
a collection unit that collects first data regarding individual behavior, second data regarding interpersonal interaction, and third data regarding a current institution in a social group,
an individual psychology diagnosis unit that diagnoses at least one of the first data and the second data under the current institution, and
an intervention unit that updates at least one of the first data and the second data by intervening in at least one of the individual behavior and the interpersonal interaction based on the diagnosis result for at least one of the first data and the second data; and
a second system including
an option generation unit that generates a plurality of options for a future institution in the social group based on the updated at least one of the first data and the second data, and the third data,
a consensus building unit that supports consensus building of the social group on the plurality of generated options, and
an update unit that updates the institution in the social group based on options agreed upon by the social group.

2. The information system according to claim 1, wherein
the individual psychology diagnosis unit obtains an individual utility value function and a social normative value function by diagnosing the first data and estimates an intersection point of the utility value function and the social normative value function as a value for the individual behavior.

3. The information system according to claim 2, wherein
the intervention unit intervenes in the at least one of the individual behavior and the interpersonal interaction if the value for the individual behavior estimated by the individual psychology diagnosis unit deviates from normative standards under the current institution.

4. The information system according to claim 1, wherein
the intervention unit intervenes in the at least one of the individual behavior and the interpersonal interaction if individual fairness deviates from a fairness standard under the current institution.

5. The information system according to claim 1, wherein
the intervention unit uses at least one of display, level meter, speaker, light, vibrator, and diffuser as a user interface to intervene in the at least one of the individual behavior and the interpersonal interaction if individual fairness deviates from a fairness standard under the current institution.

6. The information system according to claim 5, wherein
the intervention unit displays on the display a result of comparison between the individual behavior and the individual behavior of others under the current institution to intervene in the at least one of the individual behavior and the interpersonal interaction.

7. The information system according to claim 1, wherein
the second system includes a group state diagnosis unit that collects at least one of the second data and the third data, and diagnoses whether the current state of the social group is in equilibrium based on the at least one of the second data and the third data, and
the option generation unit generates a plurality of options for the future institution of the social group if the current state of the social group is diagnosed as not in equilibrium.

8. The information system according to claim 7, wherein
in the second system,
the group state diagnosis unit predicts whether or not the future state of the social group will be in equilibrium, and
the option generation unit generates a plurality of options for the future institution of the social group if the future state of the social group is predicted not to be in equilibrium.

9. The information system according to claim 1, wherein
the individual psychology diagnosis unit of the first system diagnoses at least one of the first data and the second data under the updated institution based on the third data updated by the fact that the institution of the social group is updated by the update unit of the second system.
